(19) United States
(12) Patent Application Publication
(10) Pub. No.: US 2014/0004615 A1
(43) Pub. Date: Jan. 2, 2014

(54) KIT FOR MARKING AND/OR DETECTING ALTERATION OF FUEL AND ITS METHOD THEREOF

(71) Applicants: Timothy George Wilkinson, Middlethorpe (GB); Robert Deva Arul Paulmer, Singapore (SG)

(72) Inventors: Timothy George Wilkinson, Middlethorpe (GB); Robert Deva Arul Paulmer, Singapore (SG)

(73) Assignee: DECIPHER PTE. LTD., SINGAPORE (SG)

(21) Appl. No.: 14/005,237

(22) PCT Filed: Jan. 27, 2012
(86) PCT No.: PCT/SG12/00026

§ 371 Date: Sep. 13, 2013

(30) Foreign Application Priority Data
Mar. 16, 2011 (SG) 201101949-4

(51) Int. Cl. G01N 33/22

(52) U.S. Cl. CPC G01N 33/22
USPC 436/111; 44/300; 422/70; 422/82.05

(57) ABSTRACT

There is provided at least a kit for marking, monitoring and/or detecting alteration of a fuel comprising: (i) at least one marker; and (ii) at least one substance, wherein the substance is capable of interacting with at least one laundering agent. In particular, the kit may comprise a substance which is capable of interacting with at least one laundering agent to protect and/or prevent the marker from being absorbed, adsorbed, or partially or completely removed by the laundering agent. There is also provided a method of marking fuel, the method comprising marking a fuel with: (i) at least one marker; and (ii) at least one substance, wherein the substance is capable of interacting with at least one laundering agent.

### FIELD OF INVENTION

The present invention relates to a kit for marking and detecting alteration of fuel and its method thereof.

### BACKGROUND OF THE ART

Fuel products are often taxed by the governments of countries to generate revenues to support and stimulate their economy. In addition, Governments will often subsidize the cost of essential products. In many countries, fuel is subsidized either to help the poor or to protect particular sectors of industry, for example, agricultural fuel will get a subsidy.

Fuels from different sources may be mixed together to obscure the origin of one or more of the fuels. Fuels from different sources may be, for example, differentially priced fuels, such as taxed fuel and subsidized fuel or tax-free fuel, or different grades or types of fuel, such as kerosene, diesel fuel, low-octane gasoline, high-octane gasoline, etc. In certain cases, higher priced fuel is mixed with lower priced fuel (e.g., lower grade fuel) or adulterants such as solvents. Fuel can be differentially priced for a variety of reasons. In some countries, liquid fuel such as diesel fuel, kerosene, and liquefied petroleum gas is subsidized or sold below market rates to provide more widespread access to resources. The subsidized fuel can be mixed illegally with non-subsidized fuel product, which means that the fuel companies and the countries' government lose legitimate sales or taxes and the subsidy does not reach its intended target.

In some cases, subsidized fuel is purchased and then re-sold, sometimes illegally, at a higher price. For example, subsidized fuel can be purchased and then mixed with other fuel to disguise the origin of the subsidized fuel.

In certain cases, markers placed in subsidized fuel for authentication are at least partially removed to disguise the origin of the fuel (i.e. laundering of the marker). Laundering of the markers in the fuel is a major problem. Current anti-laundering strategies rely on selecting markers that are resistant to laundering by strong acids and alkalis and common absorbents (such as clay and active carbon). However, complete protection of these markers from laundering is impossible to achieve in practice and as a result, it still poses as one of the existing problem for current anti-laundering strategies.

The present invention seeks to address the deficiencies of current anti-laundering strategies.

### SUMMARY OF INVENTION

The present invention relates to a kit for marking, monitoring and/or detecting alteration of liquid, and its methods thereof for marking of liquid. The present invention also relates to a method of detecting alteration of liquid. For example, the liquid may be a fuel, solvent, petrochemical or the like, and/or any combination thereof.

In particular, the present invention provides a new kit for marking, monitoring and/or detecting alteration of a fuel and/or solvent. The invention also relates to a method of marking fuel and/or solvent. Further, the present invention provides a method of monitoring, testing and/or detecting alteration of a first fuel with a second fuel and/or solvent.

According to a first aspect, the invention relates to a kit for marking, monitoring and/or detecting alteration of a fuel and/or solvent comprising:
at least one marker (S); and
at least one substance (A), wherein the substance (A) is capable of interacting with at least one laundering agent.

In the kit according to the invention, substance (A) is capable of interacting with at least one laundering agent to protect and/or prevent marker (S) from being absorbed, adsorbed, or partially or completely removed by the laundering agent.

In the kit according to the invention, substance (A) is capable of interacting with at least clay, carbon, active carbon, charcoal, active charcoal, paper filter, micropore filter, straw, silica, silica gel, molecular sieve and/or adsorbent material or a mixture thereof.

According to a particular aspect, the invention relates to a fuel and/or solvent comprising:
at least one marker (S); and
at least one substance (A), wherein substance (A) is capable of interacting with at least one laundering agent; and
optionally, at least one marker (C), wherein the marker (C) is a quantitative marker.

According to another aspect, the invention relates to a method of marking fuel and/or solvent, the method comprising:
marking a fuel and/or solvent with:
at least one marker (S); and
at least one substance (A), wherein the substance is capable of interacting with at least one laundering agent.

In particular, the method of marking fuel according to the invention comprises marking a first fuel and/or solvent and a second fuel to allow detection of a mixture of the first fuel and/or solvent and second fuel, the method comprising:
(I) marking at least one first fuel and/or solvent with:
   (i) at least one marker (S); and
   (ii) at least one substance (A), wherein the substance is capable of interacting with at least one laundering agent; and
(II) marking at least one second fuel with at least one marker (C), wherein marker (C) is a quantitative marker.

According to another aspect, the present invention relates to a method of monitoring, testing and/or detecting alteration of a first fuel with a second fuel and/or solvent, the second fuel and/or solvent comprising: (i) at least one marker (S); and (ii) at least one substance (A), wherein the substance (A) is capable of interacting with at least one laundering agent; the method comprising:
(I) testing the fuel for the presence of marker (S), wherein the presence of marker (S) in the fuel is indicative that the fuel is altered; or
(II) testing the fuel for the presence of substance (A), wherein substance (A) is capable of being detected, and the presence of substance (A) is indicative that the fuel is altered; or
(II) testing the fuel for the presence of at least one laundering agent.

According to a particular aspect, the invention further relates a method of monitoring, testing and/or detecting alteration of a fuel, the fuel comprising: (i) at least one marker (S); and (ii) at least one substance (A), wherein the substance (A) is capable of interacting with at least one laundering agent; the method comprising:
(I) testing the fuel for the presence of marker (S), wherein the absence of marker (S) in the fuel is indicative that the fuel is altered; or
(II) testing the fuel for the presence of substance (A), wherein substance (A) is capable of being detected, the absence of substance (A) is indicative that the fuel is altered.

### BRIEF DESCRIPTION OF THE FIGURES

The accompanying figures, which are incorporated in and constitute a part of this specification, illustrate aspects of the invention and, together with a general description of the invention given above, and the detailed description given below, serve to explain the invention.

FIG. 1 is a schematic diagram illustrating a particular aspect of the invention. In particular, it shows a first fuel (eg, unsubsidized fuel) having no marker (S) and/or substance (A) present in the first fuel and a second fuel and/or solvent (eg, subsidized fuel and/or solvent) having marker (S) and substance (A) present in the second fuel and/or solvent.

FIG. 2 is a schematic diagram illustrating a particular aspect of the invention. In particular, it shows a first fuel (eg, unsubsidized fuel) having no marker (S), marker (C) and/or substance (A) present in the first fuel and a second fuel and/or solvent (eg, subsidized fuel and/or solvent) having marker (C), marker (S) and substance (A) present in the second fuel and/or solvent.

FIG. 3 is a schematic diagram illustrating a particular aspect of the invention. In particular, it shows a first fuel (eg, unsubsidized fuel) having marker (C) present in the first fuel and a second fuel and/or solvent (eg, subsidized fuel and/or solvent) having marker (C), marker (S) and substance (A) present in the second fuel and/or solvent.

FIG. 4 is a flowchart illustrating a particular aspect of the invention. In particular, it exemplifies a method of monitoring, testing and/or detecting of a fuel having marker (S) and substance (A) present in the fuel.

FIG. 5 is a flowchart illustrating a particular aspect of the invention. In particular, it exemplifies a method of monitoring, testing and/or detecting of a fuel having marker (C), marker (S) and substance (A) present in the fuel.

### DETAILED DESCRIPTION

The present invention relates to a kit for marking, monitoring and/or detecting alteration of liquid, and its methods thereof for marking of liquid. The present invention also relates to a method of detecting alteration of liquid. For example, the liquid may be a fuel, solvent, petrochemical or the like, and/or any combination thereof.

In particular, the present invention relates to a kit for marking, monitoring and/or detecting alteration of fuel and/or solvent, and its methods thereof for marking of fuels and/or solvents that may be potentially altered or mixed to allow for detection of altered fuels. The present invention also relates to a method of detecting alteration of fuel, in particular, detecting alteration of a first fuel with a second of further fuel and/or solvent. More in particular, the present invention relate to a kit and/or a method for marking, monitoring and/or detecting alteration of fuel and/or solvent, wherein the kit and/or the method may comprise at least one substance (A) which is capable of interacting with at least one laundering agent to protect and/or prevent at least one marker (S) from being absorbed, adsorbed, or partially or completely removed by the laundering agent.

For the purposes of the present invention, the terms “alteration of fuel” or “altered fuel” or “fuel is altered” are understood to mean a fuel that has been mixed, diluted, and/or adulterated. A fuel can altered by being mixed, diluted, and/or adulterated with one or more other fuels, solvents, petrochemicals and/or any combination thereof.

The term ‘fuel’ used herein the present invention is understood to mean any hydrocarbons, petroleum based products, biofuels, fossil fuels including, but not limited to, diesel, gasoline, kerosene, and stove oil.

The term ‘petrochemicals’ used herein the present invention is understood to mean any chemical products derived from petroleum. In particular, petrochemical may be classified into olefins including ethylene and propylene, and aromatics including benzene, toluene and xylene isomers.

The term ‘solvent’ used herein the present invention is understood to mean a liquid, which dissolves another liquid, solid, or gas (particularly fuel), resulting in a solution that is soluble in a certain volume of the solvent. In particular, the solvent may be a polar or a non-polar solvent. More particularly, the solvent may comprise an organic solvent. Examples of solvents include, but are not limited to, hexane, ethyl acetate, methanol, pentane, diethyl ether, and the like.

Further, a fuel and/or solvent can be treated or “laundered” in an attempt to remove identifying features such as markers from the fuel and/or solvent (e.g., to disguise the origin of the fuel, the amount of tax paid on the fuel, etc.) before it is mixed with another fuel, solvent or petrochemical to form an altered fuel. The terms ‘laundered’ and ‘treated’ are both used interchangeably and are both understood to mean the partial or complete removal of at least one identifying feature(s) or characteristic(s) of the fuel. For example, a fuel is laundered or treated to remove partially or completely at least one marker which has been predisposed in the fuel. In particular, the fuel may also be laundered by a second fuel, solvent, petrochemical or the like. The term laundering agent is understood to mean any substance, chemical or compound which is capable of partially or completely removing any identifying feature or characteristic of the fuel, particularly the partial or complete removal of a marker in a fuel.

The term “subsidized fuel” and/or “subsidized solvent” used herein the present invention is understood to mean a fuel and/or a solvent where part of the full cost is paid for (subsidized) by the Government or other body.

According to a first aspect, the present invention relates to a kit for marking, monitoring and/or detecting alteration of a fuel and/or solvent comprising:
(i) at least one marker (S); and
(ii) at least one substance (A), wherein the substance (A) is capable of interacting with at least one laundering agent.

More in particular, in the kit according to the present invention, substance (A) is capable of interacting with at least one laundering agent to protect and/or prevent marker (S) from being absorbed, adsorbed, or partially or completely removed by the laundering agent.

A marker is defined for the purpose of the present invention as any substance, chemical or compound which can be dissolved in a fuel and/or solvent to be identified, and then subsequently detected by any suitable analytical techniques known in the field of detection of fuel markers. A marker may be concealed and/or hidden (for example, not visually detectable by the eye, or a third party have no knowledge of the presence of the marker). Alternatively, a marker may be open to view and/or knowledge (for example, a visual dye which is easily recognizable by the naked eye). Characteristics of common suitable markers for liquid petroleum products include, but not limited to, (i) being entirely foreign to the petroleum products; (ii) can be supplied as highly concentrated solutions in petroleum-compatible solvents; (iii) are not obscured by unstable natural components of the petroleum products; (iv) are measurable over the storage life of the marked petroleum product; (v) have identities which can be analyzed by standard analytical techniques; and (vi) are easily detected by simple on-site test and/or in the laboratory.

In particular, a marker is detectable by any one of the following analytical techniques, but not limited to: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy. Markers may also include colour dyes or covert chemicals which can be extracted from the fuel and/or solvent and which give a colour reaction visible to the naked eye when treated with a reagent such as acid or alkali.

Any suitable substance, chemical or compound may be used for the purpose of a marker as described herein. Examples of compounds which are suitable for use as markers are, but not limited to:
visual dyes, such as Solvent Red 24, as described in WO 2010/039152, which is incorporated herein by reference;
substituted 1,4-hydroxyanthraquinones for example, 2-(methoxyethoxypropylamino)-1,4-dihydroxyanthraquinone, 2-(butoxypropylamino)-1,4-dihydroxyanthraquinone etc, as described in U.S. Pat. No. 4,735,631, which is incorporated herein by reference;
furfural, diphenylamine and radioactive materials, as described in U.S. Pat. No. 4,735,631, which is incorporated herein by reference;
1-(amino)-3-(alpha or beta naphthylamino)-propanes, for example, 1-(4-morpholino)-3-(alpha naphthylamino)-propane and 1-(4-morpholino)-3(beta naphthylamino)-propane, as described in U.S. Pat. No. 4,209,302, which is incorporated herein by reference;
N-Ethyl-N-[2-[1-(2-methylpropoxy)ethoxy]ethyl]-4-phenyldiazenylaniline (Euromarker Solvent Yellow 24), as described in WO 2010/039152;
near infrared fluorescent compounds selected from the classes of phthalocyanines, 2,3-naphthalocyaninessquaraines (squaric acid derivatives) and croconic acid derivatives as described in U.S. Pat. No. 5,525,516, which is incorporated herein by reference;
markers that are synthesized by azo coupling of an appropriately substituted aniline to an alpha-naphthylamine, for example, 4-(2-methoxyphenylazo)-1-(3-methoxypropyl-amino)naphthalene, as described in U.S. Pat. No. 5,490,872, which is incorporated herein by reference;
coumarine (1,2-benzopyrone) which may be used to tag kerosene, as described in U.S. Pat. No. 5,980,593, which is incorporated herein by reference;
chemicals described as linear or branched chain alkyl acid esters of 7-hydroxy-4-methylcoumarin (4-methylumbelliferone), as described in U.S. Pat. No. 5,980,593, which is incorporated herein by reference;
general class of chemicals described as phenylazophenols, as described in U.S. Pat. No. 5,156,653, which is incorporated herein by reference.

In a particular aspect of the present invention, marker (S) may be selected from the group consisting of: anthraquinone, anthraquinone dye(s), diazo dye(s), furfural (furan-2-carbaldehyde), furfural (furan-2-carbaldehyde) derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68 (2-ethyl-N-(2-ethylhexyl)hexan-1-amine), CI Solvent Red 161, CI Solvent Red 164 (1-[[4-[phenylazo]-phenyl]azo]-2-naphthol), CI Solvent Red 175, CI Solvent Red 208, CI Solvent Red 165, CI Solvent Blue, CI Solvent Blue 4 (alpha,alpha-Bis[4-(dimethylamino)phenyl]-4-(phenylamino)naphthalene-1-methanol), CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124 (N-Ethyl-N-[2-[1-(2-methylpropoxy)ethoxy]ethyl]-4-phenyldiazenylaniline), CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivatives, analogues or any mixture thereof.

In a particular aspect of the present invention, marker (S) may be a quantitative marker or qualitative marker.

A qualitative marker is defined for the purpose of the present invention as any marker, wherein only the detection of the presence (or absence) of the marker is required using any suitable analytical techniques known in the art. Determination of the concentration of marker quantitatively is not necessary is not essential for a qualitative marker.

A quantitative marker is defined for the purpose of the present invention as any marker whose concentration can be detected quantitatively by any suitable standard analytical techniques known in the art. A quantitative marker as used herein may be a marker with varying degree of quantification, i.e. the quantitative marker may be capable of being fully or partially quantified by any of the standard analytical techniques known in the art. A quantitative marker includes, but not limited to, a marker whose concentration range can only be determined and quantified partially using any suitable standard analytical techniques known in the art.

The concentration of a quantitative marker (S) is to be determined quantitatively by any suitable analytical techniques known in the art. In particular, a quantitative marker (S) is to be detectable and its concentration needs to be determined quantitatively by any one of the following analytical techniques, but not limited to: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy. Marker (S) may also include colour dyes or covert chemicals which can be extracted from the fuel and which give a colour reaction visible to the naked eye when treated with a reagent such as acid or alkali or the like. The terms ‘determined’, ‘determination’, ‘detecting’ and ‘detectable’ as described herein the present invention may be used interchangeably for the purposes of the present invention.

Testing for the presence of a quantitative marker (S) in a fuel and/or solvent may be achieved on-site, for rapid determination, or in a laboratory. In particular, concentration of the quantitative marker (S) in a fuel and/or solvent can be assessed by absorption spectroscopy with ultraviolet, visible, or infrared radiation, in which absorption of radiation by the sample is proportional to the concentration of the marker in the sample. An example of the use and detection of quantitative markers is described in U.S. Pat. No. 5,525,516 to Krutak et al., which is incorporated herein by reference.

Quantitative marker (S) may be used to detect dilution, i.e. the decrease in concentration of marker (S), caused by mixing, for example, a fuel having a desired predetermined concentration of the quantitative marker (S) together with an adulterant such as a solvent (which does not contain the marker (S)).

An adulterant is defined for the purpose of the present invention as any substance, chemical or compound which should not be contained within other substances (eg. fuels for legal or other reasons). Adulterants may be intentionally added to more expensive substances to increase visible quantities and reduce manufacturing costs.

In a particular embodiment, as little as 5% is detectable with the use of the quantitative marker (S). More preferably, as little as 1% dilution is detectable with the use of the quantitative marker (S).

In a further aspect of the present invention, marker (S) includes being any substance, chemical or compound which is completely or partially resistant or not resistant to at least one laundering agent. The degree in which a particular marker (S) may be removed in a fuel and/or solvent varies with different laundering agents. For example, a particular marker (S), subject to its resistance to a particular laundering agent may be completely or partially removed or not removed at all by that particular laundering agent.

A laundering agent is defined for the purpose of the present invention as any substance, chemical or compound which is capable of partially or completely removing any identifying feature or characteristic of the fuel and/or solvent. Examples of laundering agents are, but not limited to, strong acids, alkalis or absorbents such as clay, carbon, active carbon, charcoal, active charcoal, paper filters, straw, microfilters, silica, silica gel, molecular sieves and adsorbent materials. For the purpose of the present invention, a molecular sieve may be defined as a material containing tiny pores of a precise and uniform size that is used as an adsorbent for gases and liquids. Molecular sieves may consist of aluminosilicate minerals, clays, porous glasses, microporous charcoals, zeolites, active carbons, or synthetic compounds that have open structures through which small molecules can diffuse. An example of molecular sieve is, but not limited to zeolites.

Substance (A) is defined for the purpose of the present invention as any chemical, chemical compound, organic or inorganic substance of a particular molecular identity, including any combination of such substances occurring in whole or in part as a result of a chemical reaction or occurring in nature, which is capable of interacting with any one of the following laundering agents: clay, active charcoal, charcoal, carbon, active carbon, paper filters, straw, micropore filters, silica, silica gel, molecular sieves, absorbent materials, adsorbent materials and a mixture thereof. Substance (A) may be a polar chemical, chemical compound or substance.

In particular, substance (A) is capable of being absorbed, adsorbed, or partially or completely removed by interaction with at least one of the following laundering agents: clay, active charcoal, charcoal, carbon, active carbon, paper filters, straw, micropore filters, silica, silica gel, molecular sieves, absorbent materials, adsorbent materials and a mixture thereof.

In particular, the term “interacting”, ‘interaction’ and ‘interact’ may be used interchangeably for the purposes of the present invention. The term ‘interacting’ as used herein the present invention is understood to mean a kind of action that occurs wherein two or more substances, matter, compounds and/or objects have an effect(s) upon each other. In particular, the effect, as a result of the interaction, may be a physical effect or a chemical effect. For example, ‘interacting’ may be understood to mean absorbing, adsorbing, or partially or completely removed, for the purposes of the present invention. Preferably, substance (A) is more readily absorbed, adsorbed, or partially or completely removed, in place of marker (S), by any one of the following laundering agents: clay, active charcoal, charcoal, carbon, active carbon, paper filters, straw, micropore filters, silica, silica gel, molecular sieves, adsorbent materials or a mixture thereof. In particular, substance (A) is being preferentially absorbed by clay, active charcoal, charcoal, carbon, active carbon, paper filters, straw, micropore filters, silica, silica gel, molecular sieves, absorbent materials, adsorbent materials or any mixture thereof, with respect to marker (S).

In an attempt to launder at least one marker (S) which was predisposed in a fuel, laundering agents are added into the fuel so as to launder (i.e. to partially or completely remove) the marker (S). The effect of adding substance (A) into a fuel, together with marker (S), is to protect and/or prevent marker (S) from being absorbed, or partially or completely removed by interaction with any one of the following laundering agents: clay, active charcoal, charcoal, carbon, active carbon, paper filters, straw, micropore filters, silica, silica gel, molecular sieves, absorbent materials, adsorbent materials or any mixture thereof, which are added to the fuel with the intention of laundering the markers.

In particular, the substance (A) added to the fuel is complementary to the marker (S) such that substance (A) is always more readily absorbed, adsorbed, or partially or completely removed by interaction with the laundering agents, than marker (S). Substance (A) acts as a sacrificial protection substance, chemical or compound which is being absorbed, in place of marker (S), by the laundering agents.

In a particular aspect of the invention, substance (A) may be a very polar chemical, chemical compound or substance. In particular, substance (A) is a chemical, chemical compound or substance which is more polar than marker (S).

In a particular aspect of the present invention, substance (A) may be selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivatives, analogues or mixture thereof.

Substance (A) and marker (S) may be selected from the same group of chemicals, compounds or substances as described herein. However, substance (A) and marker (S) are not the same when both are being used together (i.e. both are present) in any particular aspect of the present invention.

In particular, substance (A) may further be capable of being detected. In particular, substance (A) is capable of being detected, and optionally whose concentration is capable of being determined quantitatively, by any one of the standard analytical methods such as the following analytical techniques, but not limited to: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy. Substance (A) may also include colour dyes or covert chemicals which can be extracted from the fuel and which give a colour reaction when treated with a reagent such as acid or alkali.

In the kit according to the present invention, the kit may further comprise at least one marker (C), wherein marker (C) is a quantitative marker. For the purpose of the present invention, the definitions and examples of Marker (C) are the same as that of marker (S) as previously described herein.

In a particular aspect of the present invention, marker (C) may be selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

Marker (C), marker (S) and substance (A) may be selected from the same group of chemicals, compounds or substances as described herein. However, marker (C), marker (S) and substance (A) are not the same with respect to each other when they are being used together (i.e. when (C), (S) and (A) are all present) in any particular aspect of the present invention.

Marker (C) includes being any substance, chemical or compound which is completely or partially resistant or not resistant to at least one laundering agent. The degree in which a particular marker (C) may be removed in a fuel varies with different laundering agents. For example, a particular marker (C), subject to its resistance to a particular laundering agent may be completely or partially removed or not removed at all by that particular laundering agent.

Marker (S) may be more resistant to at least one laundering agent than marker (C). In particular, marker (S) is more resistant to partial or complete removal by at least one laundering agent, as compared to marker (C). Marker (S) and Marker (C) may be subject to the same or different laundering agent. Preferably, marker (S) is more resistant to partial or complete removal by at least one same laundering agent, as compared to marker (C).

In a particular aspect, the kit of the present invention comprises a quantitative marker (C) and a quantitative marker (S), the quantitative marker (S) is different from the quantitative marker (C). In particular, the kit comprises two distinct quantitative markers, marker (S) and marker (C) respectively.

According to another embodiment, the kit of the present invention comprises at least one marker (S), at least one substance (A), and optionally at least one marker (C), which are provided in the form of a mixture. Marker (S), substance (A) and optionally marker (C) may be in the form of a homogenous or heterogeneous mixture. The mixture may be in the form of a liquid mixture, with a predetermined amount of each of the respective marker (S), substance (A), and optionally marker (C). The mixture may also be in the form of a tablet or capsule form, with a predetermined amount of each of the respective marker (S), substance (A), and optionally marker (C).

According to a particular embodiment, the kit of the invention comprises (i) at least one marker (C), one marker (S) and one substance (A) for addition to a first fuel and/or solvent; and (ii) at least one marker (C) for addition to a second fuel; both markers (C) being the same. In particular, there is at least one common marker (C) in the first fuel and/or solvent and the second fuel, the common marker (C) being a quantitative marker. Marker (C) having a predetermined concentration range is added to both the first fuel and/or solvent and the second fuel, the predetermined concentration range of marker (C) in the first fuel and/or solvent may or may not be the same as the predetermined concentration range of maker (C) in the second fuel. In particular, the first fuel may be a subsidized fuel and/or solvent and the second fuel may be an unsubsidized fuel. The purpose of the kit of the invention is for marking, monitoring and/or detecting alteration of a fuel, particularly whether an unsubsidized fuel has been altered with a subsidized fuel or vice versa, or whether a particular fuel has been altered by diluting with solvent and/or petrochemical.

In some embodiments, the kit of the invention may comprise one or more additional markers, such as quantitative markers, qualitative markers or any combination thereof.

Fuel and Fuel Composition

According to a particular aspect, the present invention relates to a fuel and/or solvent comprising:
(i) at least one marker (S); and
(ii) at least one substance (A), wherein substance (A) is capable of interacting with at least one laundering agent; and
optionally, (iii) at least one marker (C).

According to one aspect, the present invention relates to a fuel and/or solvent, wherein the fuel and/or solvent comprises optionally at least one marker (C), the at least one marker (C) is a quantitative marker.

The fuel and/or solvent according to the invention, wherein the fuel and/or solvent is subject to monitoring, marking and/or detecting alteration of the said fuel and/or solvent with any laundering agents or with another different fuel and/or solvent. For example, the fuel may be a subsidized diesel by the government or unsubsidized kerosene. The fuel and/or solvent of the invention comprising the marker (S), substance (A) and optionally marker (C) is used for monitoring and detecting the presence of this particular fuel, particularly, to detect and monitor any unauthorized circulation and distribution of this particular marked fuel and/or solvent. The fuel and/or solvent of the invention comprises at least one substance (A), wherein substance (A) is capable of interacting with at least one laundering agent. The laundering agent is selected from the group consisting of: clay, carbon, active carbon, charcoal, active charcoal, paper filter, straw, micropore filters, silica, silica gel, molecular sieves, absorbent materials, adsorbent materials and any mixture thereof.

In particular, substance (A) is capable of being absorbed, adsorbed, or partially or completely removed by interaction with at least one of the following laundering agents: clay, active charcoal, charcoal, carbon, active carbon, paper filters, straw, micropore filters, silica, silica gel, molecular sieves, absorbent materials, adsorbent materials and a mixture thereof.

In an attempt to launder at least one marker (S) which was predisposed in a fuel, laundering agents are added into the fuel so as to launder (i.e. to partially or completely remove) the marker (S). The effect of adding substance (A) into a fuel, together with marker (S), is to protect and/or prevent marker (S) from being absorbed, or partially or completely removed by interaction with any one of the following laundering agents: clay, active charcoal, charcoal, carbon, active carbon, paper filters, straw, micropore filters, silica, silica gel, molecular sieves, absorbent materials, adsorbent materials or any mixture thereof, which are added to the fuel with the intention of laundering the markers.

In particular, the substance (A) added to the fuel is complementary to the marker (S) such that substance (A) is always more readily absorbed, adsorbed, or partially or completely removed by interaction with the laundering agents, than marker (S). Substance (A) acts as a sacrificial protection substance, chemical or compound which is being absorbed, in place of marker (S), by the laundering agents.

In particular, the fuel and/or solvent of the invention comprises at least one substance (A), wherein substance (A) is selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

In particular, the fuel and/or solvent of the invention comprises at least one marker (S), wherein marker (S) is a selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

In particular, the fuel and/or solvent of the invention may comprise at least one marker (C), wherein marker (C) is a selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

Marker (S), marker (C) and substance (A) may be selected from the same group of chemicals, compounds or substances as described herein. However, marker (S), marker (C) and substance (A) are not the same with respect to each other when they are being used together (i.e. when (S), (C) and (A) are all present) in any particular aspect of the present invention.

The terms ‘marker (C)’, marker (S), ‘quantitative marker’, ‘substance (A)’ and ‘laundering agent’ are as defined in the description herein.

According to another aspect, the present invention relates to a fuel composition comprising a fuel and/or solvent, wherein the fuel and/or solvent comprises (i) at least one marker (S); and (ii) at least one substance (A), wherein substance (A) is capable of interacting with at least one laundering agent; and optionally, (iii) at least one marker (C), wherein the marker (C) is a quantitative marker.

The term fuel composition used herein is defined as a mixture of fuels, solvents, and/or petrochemicals. For example, a fuel composition may comprise a mixture of diesel and kerosene, or a mixture of gasoline and kerosene, or a mixture of government tax-subsidized diesel and non-tax-subsidized diesel.

More particularly, the fuel composition of the invention may comprise a mixture of at least a one fuel comprising a marker (C) and another fuel and/or solvent comprising (i) at least one marker (S); and (ii) at least one substance (A), wherein substance (A) is capable of interacting with at least one laundering agent; and optionally, (iii) at least one marker (C), wherein the marker (C) is a quantitative marker.

Method of Marking Fuel

According to one aspect, the present invention relates to a method of marking fuel and/or solvent comprising marking a fuel and/or solvent with (i) at least one marker (S); and (ii) at least one substance (A), wherein the substance is capable of interacting with at least one laundering agent.

In particular, substance (A) is capable of being absorbed, adsorbed, or partially or completely removed by interaction with at least one of the following laundering agents: clay, active charcoal, charcoal, carbon, active carbon, paper filters, straw, micropore filters, silica, silica gel, molecular sieves, absorbent materials, adsorbent materials and a mixture thereof.

In an attempt to launder at least one marker (S) which was predisposed in a fuel, laundering agents are added into the fuel so as to launder (i.e. to partially or completely remove) the marker (S). The effect of adding substance (A) into a fuel, together with marker (S), is to protect and/or prevent marker (S) from being absorbed, or partially or completely removed by interaction with any one of the following laundering agents: clay, active charcoal, charcoal, carbon, active carbon, paper filters, straw, micropore filters, silica, silica gel, molecular sieves, absorbent materials, adsorbent materials or any mixture thereof, which are added to the fuel with the intention of laundering the markers.

In particular, the substance (A) added to the fuel is complementary to the marker (S) such that substance (A) is always more readily absorbed, adsorbed, or partially or completely removed by interaction with the laundering agents, than marker (S). Substance (A) acts as a sacrificial protection substance, chemical or compound which is being absorbed, in place of marker (S), by the laundering agents.

According to another embodiment, the method of marking fuel and/or solvent as herein described in the invention, further comprises marking a fuel and/or solvent with at least one marker (C), marker (C) being a quantitative marker. In particular, a particular fuel is marked with at least one marker (S), at least one quantitative marker (C), and at least one substance (A) which is capable of interacting with at least one laundering agent. For example, the fuel may be exemplified by a subsidized or an unsubsidized fuel such as diesel or kerosene.

In a particular embodiment, the method of marking fuel and/or solvent as described herein the present invention further include a method of marking a first fuel and/or solvent and a second fuel to allow detection of a mixture of the first fuel and/or solvent and second fuel, the method comprising
(I) marking at least one first fuel and/or solvent with: (i) at least one marker (S); and (ii) at least one substance (A), wherein the substance is capable of interacting with at least one laundering agent; and
(II) marking at least one second fuel with at least one marker (C), wherein marker (C) is a quantitative marker.

In this particular embodiment, the first fuel and/or solvent may be exemplified by a subsidized fuel and/or solvent which is marked with at least one marker (S) and at least one substance (A). The second fuel may be exemplified by a non-subsidized fuel which is marked with a quantitative marker (C).

In another embodiment, the method of marking fuel and/or solvent of the invention may further include the method of marking a first fuel and/or solvent (eg, subsidized fuel and/or solvent) and a second fuel (unsubsidized fuel) to allow detection of a mixture of the first fuel and/or solvent and second fuel, the method comprising:
(I) marking at least one first fuel and/or solvent with (i) at least one marker (C), wherein marker (C) is a quantitative marker; (ii) at least one marker (S); and (iii) at least one substance (A), wherein the substance is capable of interacting with at least one laundering agent; and
(II) marking at least one second fuel with at least one marker (C); both markers (C) being the same.

In this particular embodiment, the first fuel and/or solvent may be exemplified a subsidized fuel and/or solvent which is marked with at least one marker (S), at least one quantitative marker (C) and at least one substance (A). The second fuel may be exemplified by a non-subsidized fuel which is marked with the same quantitative marker (C).

The terms ‘marker (C)’, marker (S), ‘quantitative marker’, ‘substance (A)’ and ‘laundering agent’ are as defined in the description herein.

In a particular embodiment wherein the marker (S) as described in the method of marking fuel and/or solvent of the invention is a quantitative marker and the method further comprises a quantitative marker (C), the quantitative marker (S) is different from the quantitative marker (C).

Substance (A) as described in the method of marking fuel and/or solvent of the invention is capable of interacting with at least one laundering agent. The laundering agent may be selected, but not limited to, from the group consisting of: clay, carbon, active carbon, charcoal, active charcoal, paper filter, straw micropore filter, silica, silica gel, molecular sieve, absorbent material, adsorbent material and a mixture thereof.

Substance (A) as described in the method of marking fuel and/or solvent of the invention may be further capable of being detected, and optionally whose concentration is capable of being determined quantitatively, by any one of the standard analytical methods such as the following analytical techniques, but not limited to: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy.

Preferably, substance (A) is selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue, or mixture thereof.

Markers (C), markers (S) and substance (A) are chosen such that the respective markers do not interfere with each other or mask the presence of each other. In particular, the presence of marker (C) in a fuel should not cause marker (S) in the fuel to lose its detectable characteristic or feature. Further, the markers also do not react with each other or with the fuel to be marked.

In another embodiment, marker (S) as described in the method of marking fuel and/or solvent herein for the present invention is fully or partially resistant to at least one laundering agent. In particular, the marker (S) may be partially removed or not removed at all from the fuel it is marked with.

In order to mark and detect any alteration of a fuel composition, it is desirable for both the quantitative marker (C) and marker (S) to be susceptible to laundering with at least one laundering agent. Accordingly, marker (S), marker (C) and optionally substance (A) as described in the methods of marking fuel herein the present invention may be capable of being resistant to partial or complete removal or capable of being partially or completely removed from the fuel by at least one laundering agent. Preferably, marker (S), marker (C) and optionally substance (A) are resistant to partial or complete removal or susceptible to partially or complete removal from the fuel by the one common laundering agent.

Preferably, marker (S) is more resistant to one or more laundering techniques which reduce the concentration of marker (C) in the fuel. It is advantageous when the quantitative marker (C) is more susceptible to laundering by a particular laundering agent than marker (S), such that a lower concentration of the quantitative marker (C), the presence of marker (S), or a combination thereof can be detected, and is evidence of an altered fuel. Accordingly, treatment to remove at least some amount of marker (S), results in the partial or complete removal of marker (C) from the fuel. The term ‘treatment’ is understood to mean laundering of the fuel or any one of the markers present in the fuel, including the removal of these markers in the fuel.

Preferably, marker (S) and/or marker (C) are selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

Marker (S), marker (C) and substance (A) may be selected from the same group of chemicals, compounds or substances as described herein. However, marker (S), marker (C) and substance (A) are not the same with respect to each other when they are being used together (i.e. when (C), (S) and (A) are all present) in any particular aspect of the present invention.

Marker (C), marker (S) and optionally substance (A) as described in the method of marking fuel herein the present invention is detectable by any suitable standard analytical techniques known in the art. These analytical techniques may include, but not limited to, the following: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy.

Method of Detecting Alteration of Fuel

Following the marking of fuel to be monitored, tracked or detected for alteration or laundering, the fuel may subsequently be monitored, tested, or detected for alteration of the said fuel by a method of detection as described herein the present invention. The fuel may comprise a single type of fuel diluted with a solvent and/or petrochemical, a mixture of different type fuels (eg, diesel and kerosene, or diesel and gasoline etc), or a mixture of the same type of fuel but of different grade (eg, high grade diesel and low grade diesel), or a mixture of a laundered fuel and an unlaundered fuel (eg, kerosene with markers being laundered or removed and kerosene with unlaundered or unremoved markers).

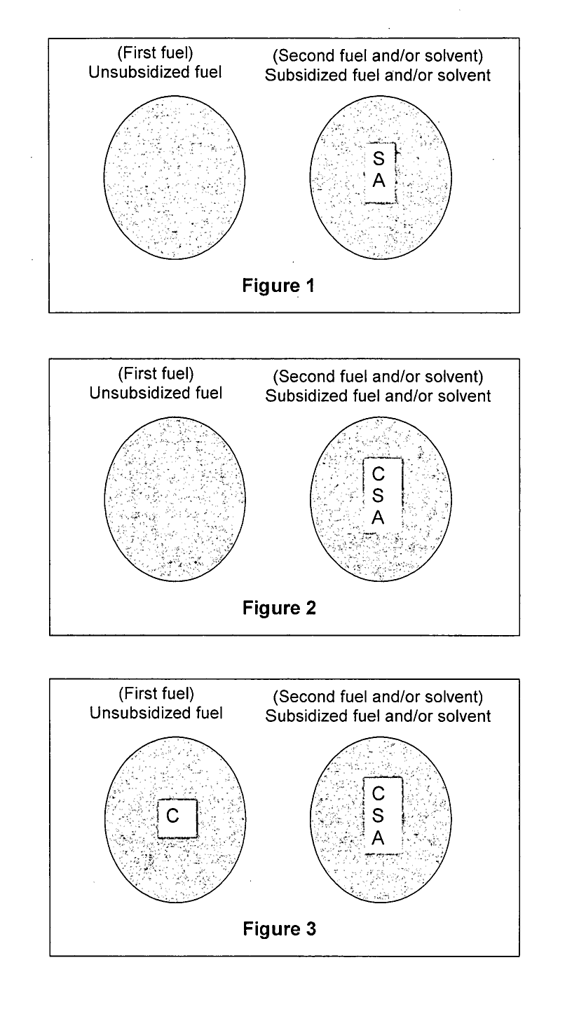

According to one aspect (as exemplified in, but not limited to, FIG. 1), the present invention relates to a method of monitoring, testing and/or detecting alteration of a first fuel (eg, unsubsidized fuel) with a second fuel and/or solvent (eg, subsidized fuel and/or solvent), the second fuel and/or solvent (eg, subsidized fuel and/or solvent) comprising (i) at least one marker (S); and (ii) at least one substance (A), wherein the substance (A) is capable of interacting with at least one laundering agent, the method comprising:
(I) testing the fuel for the presence of marker (S), wherein the presence of marker (S) in the fuel is indicative that the fuel is altered; or
(II) testing the fuel for the presence of substance (A), wherein substance (A) is capable of being detected, and the presence of substance (A) is indicative that the fuel is altered; or
(III) testing the fuel for the presence of at least one laundering agent.

In particular, substance (A) is capable of being absorbed, adsorbed, or partially or completely removed by interaction with at least one of the following laundering agents: clay, active charcoal, charcoal, carbon, active carbon, paper filters, straw, micropore filters, silica, silica gel, molecular sieves, absorbent materials, adsorbent materials and a mixture thereof.

In an attempt to launder at least one marker (S) which was predisposed in a fuel, laundering agents are added into the fuel so as to launder (i.e. to partially or completely remove) the marker (S). The effect of adding substance (A) into a fuel, together with marker (S), is to protect and/or prevent marker (S) from being absorbed, or partially or completely removed by interaction with any one of the following laundering agents: clay, active charcoal, charcoal, carbon, active carbon, paper filters, straw, micropore filters, silica, silica gel, molecular sieves, absorbent materials, adsorbent materials or any mixture thereof, which are added to the fuel with the intention of laundering the markers.

In particular, the substance (A) added to the fuel is complementary to the marker (S) such that substance (A) is always more readily absorbed, adsorbed, or partially or completely removed by interaction with the laundering agents, than marker (S). Substance (A) acts as a sacrificial protection substance, chemical or compound which is being absorbed, in place of marker (S), by the laundering agents.

The testing of the fuel may be done using any suitable analytical techniques known in the field of detection of fuel markers. Examples of suitable analytical techniques include, but not limited to: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy.

In particular, the method of monitoring, testing and/or detecting alteration of fuel is for determining whether a particular fuel is an altered fuel. The term ‘altered fuel’ is understood to mean that the fuel comprises (i) a mixture of at least a first fuel and a second fuel, (ii) a first fuel diluted with a solvent or petrochemical, or (iii) a first fuel being laundered to remove at least one marker (S) or substance (A) from the fuel, or any combination thereof.

As for the first fuel (eg, unsubsidized fuel) as described in the method of detecting alteration herein the present invention, the first fuel is not be marked with any markers (S), substance (A) or even marker (C). Accordingly, if the detection method is to determine whether a first fuel has been altered by a second fuel and/or solvent,
(i) the absence of markers (S) and substance (A) in the fuel is indicative that the first fuel has not been altered;
(ii) the presence of marker (S) or substance (A) in the fuel is indicative that the fuel has been altered;
(iii) the presence of at least one laundering agent in the fuel is indicative that the fuel has been altered.

According to one aspect (as exemplified in, but not limited to, FIG. 2), the invention relates to a method of monitoring, testing and/or detecting alteration of a first fuel (eg, unsubsidized fuel) with a second fuel and/or solvent (eg, subsidized fuel and/or solvent), the second fuel and/or solvent (eg, subsidized fuel and/or solvent) further comprising at least one quantitative marker (C) having a predetermined concentration range, wherein the method comprising testing the fuel for the presence of marker (C) in the fuel such that the presence of marker (C) in the fuel is indicative that the fuel is altered. Since the first fuel is not marked with any one of marker (S), marker (C) or substance (A),
(i) the absence of markers (S) and substance (A) and marker (C) in the fuel is indicative that the first fuel has not been altered;
(ii) the presence of marker (S), marker (C) or substance (A) in the fuel is indicative that the fuel has been altered;
(iii) the presence of at least one laundering agent in the fuel is indicative that the fuel has been altered.

According to another aspect (as exemplified in, but not limited to, FIG. 3), the first fuel (eg, unsubsidized fuel) may comprise at least one quantitative marker (C), and the second fuel and/or solvent (eg, subsidized fuel and/or solvent) comprising: (i) at least one quantitative marker (C), (ii) at least one marker (S); and (iii) at least one substance (A), wherein the substance (A) is capable of interacting with at least one laundering agent; both markers (C) being the same and at a predetermined concentration range. Accordingly, the method of monitoring, testing and/or detecting alteration of a first fuel (eg, unsubsidized fuel) with a second fuel and/or solvent (eg, subsidized fuel and/or solvent) comprises:
(I) testing the fuel for the presence of the quantitative marker (C); wherein the presence of the marker (C) in the fuel having a concentration outside the predetermined concentration range or the absence of a marker (C) in the fuel is indicative that the fuel is altered; or
(II) testing the fuel for the presence of marker (C) within the predetermined concentration range and for the presence of marker (S), wherein the presence of marker (S) in the fuel is indicative that the fuel is altered; or
(III) testing the fuel for the presence of marker (C) within the predetermined concentration range, the absence of marker (S), and the presence of substance (A) is indicative that the fuel is altered.

According to another embodiment, the first fuel (eg, unsubsidized fuel) may comprise at least one quantitative marker (C) at a predetermined concentration range, and the second fuel and/or solvent (eg, subsidized fuel and/or solvent) comprising: (i) at least one marker (S); and (ii) at least one substance (A), wherein the substance (A) is capable of interacting with at least one laundering agent. Accordingly, the method of monitoring, testing and/or detecting alteration of a first fuel (eg, unsubsidized fuel) with a second fuel and/or solvent (eg, subsidized fuel and/or solvent) comprises:
(I) testing the fuel for the presence of quantitative marker (C), wherein the presence of marker (C) in the fuel outside the predetermined concentration range or the absence of marker (C) is indicative that the fuel is altered;
(II) testing the fuel for the presence of marker (C) within the predetermined concentration range and for the presence of marker (S), wherein the presence of marker (S) in the fuel is indicative that the fuel is altered;
(III) testing the fuel for the presence of marker (C) within the predetermined concentration range, the absence of marker (S), and the presence of substance (A) is indicative that the fuel is altered.

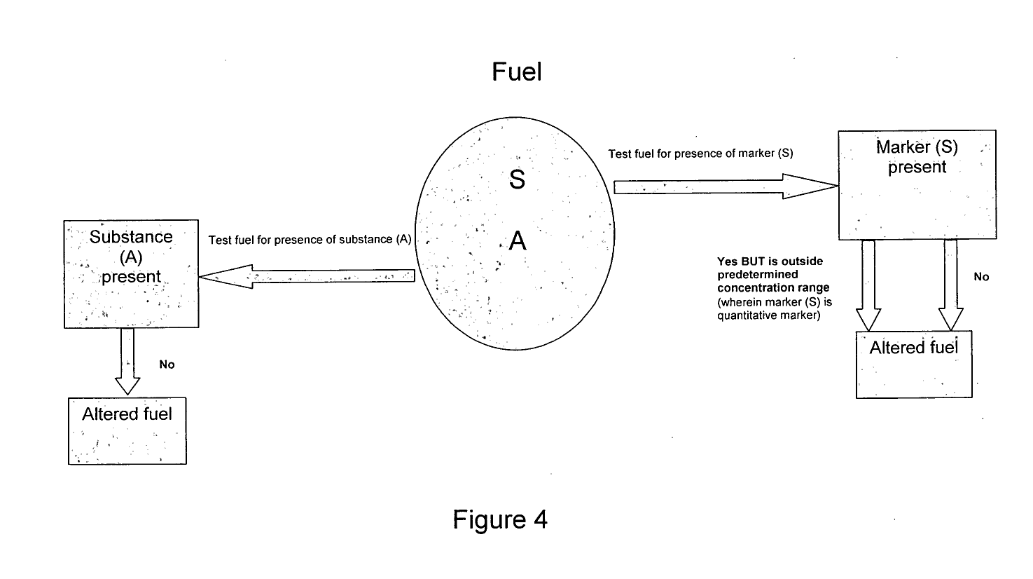

According to a particular aspect (as exemplified in, but not limited to, FIG. 4), the present invention relates to a method of monitoring, testing and/or detecting alteration of a fuel, the fuel comprising: (i) at least one marker (S); and (ii) at least one substance (A), wherein the substance (A) is capable of interacting with at least one laundering agent;
the method comprising:
(I) testing the fuel for the presence of marker (S), wherein the absence of marker (S) in the fuel is indicative that the fuel is altered; or
(II) testing the fuel for the presence of substance (A), wherein substance (A) is capable of being detected, the absence of substance (A) is indicative that the fuel is altered.

In particular, substance (A) is capable of being absorbed, adsorbed, or partially or completely removed by interaction with at least one of the following laundering agents: clay, active charcoal, charcoal, carbon, active carbon, paper filters, straw, micropore filters, silica, silica gel, molecular sieves, absorbent materials, adsorbent materials and a mixture thereof.

In an attempt to launder at least one marker (S) which was predisposed in a fuel, laundering agents are added into the fuel so as to launder (i.e. to partially or completely remove) the marker (S). The effect of adding substance (A) into a fuel, together with marker (S), is to protect and/or prevent marker (S) from being absorbed, or partially or completely removed by interaction with any one of the following laundering agents: clay, active charcoal, charcoal, carbon, active carbon, paper filters, straw, micropore filters, silica, silica gel, molecular sieves, absorbent materials, adsorbent materials or any mixture thereof, which are added to the fuel with the intention of laundering the markers.

In particular, the substance (A) added to the fuel is complementary to the marker (S) such that substance (A) is always more readily absorbed, adsorbed, or partially or completely removed by interaction with the laundering agents, than marker (S). Substance (A) acts as a sacrificial protection substance, chemical or compound which is being absorbed, in place of marker (S), by the laundering agents.

In particular, the present invention relates to the method of monitoring, testing and/or detecting alteration of a fuel, the fuel comprising: (i) at least one marker (S); and (ii) at least one substance (A), wherein the substance (A) is capable of interacting with at least one laundering agent;
the method comprising:
(I) testing the fuel for the presence of marker (S), wherein the absence of marker (S) in the fuel is indicative that the fuel is altered; or
(II) testing the fuel for the presence of substance (A), wherein substance (A) is capable of being detected, the absence of substance (A) is indicative that the fuel is altered; and
wherein the fuel is altered with at least a second or further fuel, a solvent, petrochemical or a mixture thereof.

The original unaltered fuel which has initially been marked with marker (S) and substance (A) will show the presence of the marker (S) and substance (A) respectively upon any testing of the fuel via any one of the standard analytical techniques. Accordingly, any absence of marker (S) or substance (A) in the fuel during the test would be indicative that the fuel has been altered.

In another embodiment (as exemplified in, but not limited to, FIG. 4), for the purpose of the method of monitoring, testing and/or detecting alteration of a fuel, marker (S) may be a quantitative marker. Accordingly, the method of monitoring, testing and/or detecting alteration of a fuel, the fuel comprising: (i) at least one quantitative marker (S); and (ii) at least one substance (A), wherein the substance (A) is capable of interacting with at least one laundering agent;
the method comprising:
(I) testing the fuel for the presence of marker (S), wherein the absence of marker (S) in the fuel is indicative that the fuel is altered; or
(II) testing the fuel for the presence of substance (A), wherein substance (A) is capable of being detected, the absence of substance (A) is indicative that the fuel is altered; or
(III) testing the fuel for the presence of quantitative marker (S), wherein the presence of marker (S) in the fuel having a concentration outside the predetermined concentration range is indicative that the fuel is altered.

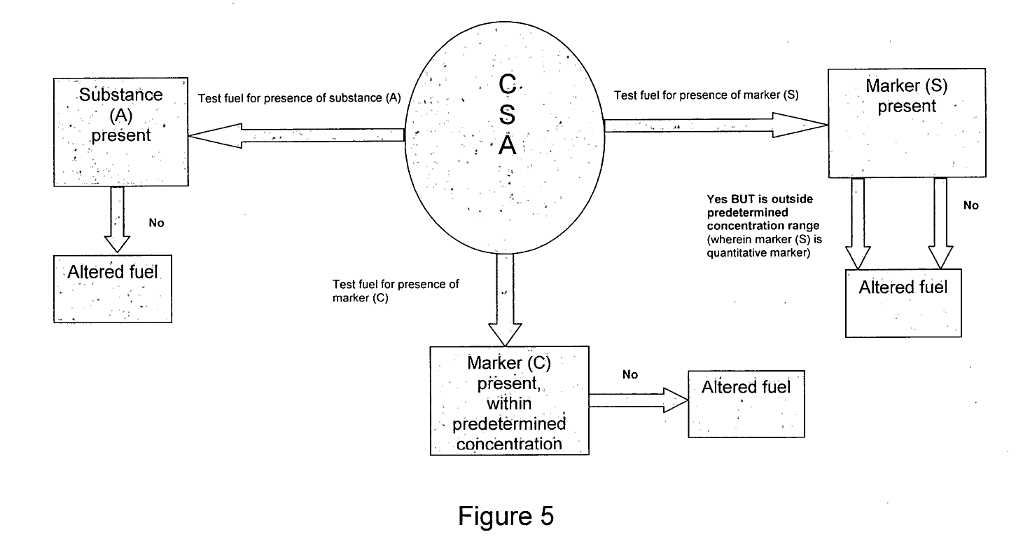

According to yet another embodiment (as exemplified in, but not limited to, FIG. 5), the fuel may further comprise at least one quantitative marker (C) having a predetermined concentration range and is different from marker (S). Accordingly, the method of monitoring, testing and/or detecting alteration of a fuel, the fuel comprising: (i) at least one marker (S); (ii) at least one quantitative marker (C) and (iii) at least one substance (A), wherein the substance (A) is capable of interacting with at least one laundering agent;
the method comprising:
(I) testing the fuel for the presence of marker (S), wherein the absence of marker (S) in the fuel is indicative that the fuel is altered; or
(II) testing the fuel for the presence of substance (A), wherein substance (A) is capable of being detected, the absence of substance (A) is indicative that the fuel is altered; or
(III) testing the fuel for the presence of quantitative marker (S), wherein the presence of marker (S) in the fuel having a concentration outside the predetermined concentration range is indicative that the fuel is altered.
(IV) testing the fuel for the presence of marker (C) in the fuel, wherein the presence of marker (C) in the fuel having a concentration outside the predetermined concentration range is indicative that the fuel is altered.

The terms ‘marker (C)’, marker (S), ‘quantitative marker’, ‘substance (A)’ and ‘laundering agent’ are as defined in the description herein.

In a particular embodiment wherein the marker (S), as described in the method of detecting alteration of fuel herein the present invention, is a quantitative marker, the quantitative marker (S) is different from the quantitative marker (C).

Substance (A) as described in the method of detecting alteration of fuel herein the present invention is capable of interacting with at least one laundering agent. The laundering agent may be selected, but not limited to, from the group consisting of: clay, carbon, active carbon, charcoal, active charcoal, paper filter, micropore filter, straw, silica, silica gel, molecular sieve, absorbent material, adsorbent material and a mixture thereof.

Preferably, substance (A) is selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

Markers (C), markers (S) and substance (A) are chosen such that the respective markers do not interfere with each other or mask the presence of each other. In particular, the presence of marker (C) in a fuel should not cause marker (S) in the fuel to lose its detectable characteristic or feature. Further, the markers also do not react with each other or with the fuel to be marked.

In another embodiment, marker (S) as described in the method of detecting alteration of fuel herein the present invention is fully or partially resistant to at least one laundering agent. In particular, the marker (S) may be partially removed or not removed at all from the fuel it is marked with.

To detect any alteration of a fuel composition, both the quantitative marker (C) and marker (S) are to be susceptible to laundering with at least one laundering agent. Accordingly, marker (S), marker (C) and optionally substance (A) as described in the methods of detecting alteration of fuel herein the present invention may be capable of being resistant to partial or complete removal or capable of being partially or completely removed from the fuel by at least one laundering agent. Preferably, marker (S), marker (C) and optionally substance (A) are resistant to partial or complete removal or susceptible to partially or complete removal from the fuel by the one common laundering agent.

Preferably, marker (S) is more resistant to one or more laundering techniques which reduce the concentration of marker (C) in the fuel. The quantitative marker (C) is more susceptible to laundering by a particular laundering agent than marker (S), such that a lower concentration of the quantitative marker (C), the presence of marker (S), or a combination thereof can be detected, and is evidence of an altered fuel. Accordingly, treatment to remove at least some amount of marker (S), results in the partial or complete removal of marker (C) from the fuel. The term ‘treatment’ is understood to mean laundering of the fuel or any one of the markers present in the fuel, including the removal of these markers in the fuel.

Marker (C), marker (S) and optionally substance (A) as described in the method of detecting alteration of fuel herein the present invention is detectable by any suitable standard analytical techniques known in the art. These analytical techniques may include, but not limited to, the following: gas chromatography (GC), gas chromatography mass spectroscopy (GCMS), high performance liquid chromatography (HPLC), x-ray diffraction, immunoassay, Raman spectroscopy, luminescence, ultraviolet spectroscopy, near infra-red spectroscopy and infra-red spectroscopy.

Preferably, marker (S) and/or marker (C) are selected from the group consisting of: anthraquinone, anthraquinone dye(s), diazo dye(s), furfural, furfural derivative(s), thiobenzotriazoles, benztriazole chemicals, paraquat, diquat, quinizarin, quinizarin derivative(s), diphenylamine, radioactive material(s), oil-soluble phenylazoaniline dye, 2,6-dicyano-2′-sulfonamido-4′-amino-azo-benzene(s), fluorene copolymer(s), rhodamine, derivatives of rhodamine, analogues of rhodamine, alkylated isodibenzanthrone, alkylated isodibenzanthrone derivative(s), cyanobenzene, 1-(4-morpholino)-3-(alpha naphthylamino)-propane, squaraines phthalocyanine(s), naphthalocyanine, CI Solvent Red, CI Solvent Red 2, CI Solvent Red 4, CI Solvent Red 68, CI Solvent Red 161, CI Solvent Red 164, CI Solvent Red 175, CI Solvent Red 208, CI Solvent Red 165, CI Solvent Blue, CI Solvent Blue 4, CI Solvent Blue 79, CI Solvent Blue 98, CI Solvent Blue 99, CI Solvent Blue 129, CI Solvent Orange 98, CI Solvent Yellow, CI Solvent Yellow 3, CI Solvent Yellow 4, CI Solvent Yellow 124, CI Solvent Yellow 143, CI Solvent Yellow 161, CI Solvent Black, CI Solvent Black 2, CI Solvent Black 7, CI Solvent Black 48, CI Solvent Brown 52, CI Solvent Green and any derivative, analogue or mixture thereof.

Marker (S), marker (C) and substance (A) may be selected from the same group of chemicals, compounds or substances as described herein. However, marker (S), marker (C) and substance (A) are not the same with respect to each other when they are being used together (i.e. when (C), (S) and (A) are all present) in any particular aspect of the present invention.

Apparatus for Monitoring, Testing and/or Detecting Alteration of at Least One Fuel

In a particular aspect, the present invention relates to an apparatus for monitoring, testing and/or detecting alteration of at least one fuel and/or solvent, the fuel and/or solvent comprising: (i) at least one marker (S); and (ii) at least one substance (A), wherein the substance (A) is capable of interacting with at least one laundering agent; the apparatus comprising at least one means for detecting the presence and/or quantity of at least maker (S) and/or substance (A).

In the apparatus according to the present invention, the fuel and/or solvent may further comprise at least one quantitative marker (C).

The terms ‘quantitative marker (C)’, ‘marker (S)’, ‘substance (A)’ and ‘laundering agent’ are as defined in the description herein.

In a particular, the present invention relates to an apparatus for monitoring, testing and/or detecting alteration of at least one fuel and/or solvent, wherein the detecting of the presence and/or quantity of marker (S), substance (A) and/or marker (C) is by the use of at least one of the following means: gas chromatography (GC), gas chromatography mass spectroscopy (GCMS), high performance liquid chromatography (HPLC), X-Ray diffraction, immunoassay, Raman spectroscopy, luminescence, ultra-violet spectroscopy, infra-red spectroscopy, near infra-red spectroscopy or any combination thereof.

Detection of Markers in Fuels Using Surface Enhanced Raman Scattering (SERS) Spectroscopy

In Raman Spectroscopy, a laser is used to excite a molecule. The intensity of the frequency difference between the molecule and the molecule affected by the laser is called the ‘Raman effect’. The frequency difference is very small and is thus enhanced by using the techniques of surface enhancement using nanoparticles (known as Surface Enhanced Raman Scattering) (SERS).

One of the methods for detected markers, particularly markers (S), markers (C) and/or substance (A) as described herein the present invention includes Surface Enhanced Raman Scattering (SERS) spectroscopy. By using SERS, the markers molecules are linked to nanoparticles (eg, silver nanoparticles) which increase the sensitivity of analysis using Raman Spectroscopy.

Nanoparticles include colloidal metal, hollow or filled nanobars, magnetic, paramagnetic, conductive or insulating nanoparticles, synthetic particles, hydrogels (colloids or bars), and the like. Colloids may be, but not limited to, silver or gold. Nanoparticles can exist in a variety of shapes, including but not limited to spheroids, rods, disks, pyramids, cubes, cylinders, nanohelixes, and nanosprings.

A marked fuel may be extracted from the fuel and mixed with a colloidal solution or gel before measurement.

Alternatively, the marker may be measured directly by being associated with a SERS active substrate prior to detection of the Raman spectrum.

Various commercially available systems/instruments can be used for detection of a Raman spectrum. Some suitable Raman spectrometers for use in the present invention include but are not limited to Raman Systems Inc., Enwave Optronics, Inc., Kaiser Optical Systems, Inc., InPhotonics, Inc., J-Y Horiba, Renishaw, Bruker Optics, Thermo Electron, Avalon, GE Ion Track, Delta Nu, Concurrent Analytical, Raman Systems, Inphotonics, Chemlmage, Jasco, Lambda Systems, Ocean Optics, SpectraCode, Savante, Real-Time Analyzers, Veeco, Witec.

Bibliographic references mentioned in the present specification are for convenience listed in the form of a list of references and added at the end of the examples. The whole content of such bibliographic references is herein incorporated by reference.

Having now generally described the invention, the same will be more readily understood through reference to the following examples which are provided by way of illustration, and are not intended to be limiting of the present invention.

### EXAMPLES

### Example 1

Properties of Markers
(I). When 1,4-Diaminoanthraquinone (1,4-diaminoanthracene-9,10-dione) [marker (S)] is treated with clay [laundering agent], 1,4-Diaminoanthraquinone is fully or partially removed or is not detectable;
(II). When 1,4-Diaminoanthraquinone [marker (S)] together with (ethyl 2-[3-(ethylamino)-6-ethylimino-2,7-dimethylxanthen-9-yl]benzoate hydrochloride) (C.I 42555) [substance (A)] is treated with clay, the (ethyl 2-[3-(ethylamino)-6-ethylimino-2,7-dimethylxanthen-9-yl]benzoate hydrochloride) [substance (A)] is preferentially removed and this “protects” 1,4-Diaminoanthraquinone [marker (S)] from the clay [laundering agent].

### Samples

Sample A: Fuel with 1 parts per million (ppm) 1,4-Diaminoanthraquinone [marker (S)];

Sample B: Fuel with 1 ppm 1,4-Diaminoanthraquinone [marker (S)] and 10 ppm (ethyl 2-[3-(ethylamino)-6-ethylimino-2,7-dimethylxanthen-9-yl]benzoate hydrochloride) [substance (A)].

Experimental Results

Effect of (ethyl 2-[3-(ethylamino)-6-ethylimino-2,7-dimethylxanthen-9-yl]benzoate hydrochloride) [substance (A)] on the detection of 1,4-Diaminoanthraquinone [marker (S)]

In this experiment, the marker (S), 1,4-Diaminoanthraquinone was extracted from the fuel with an alkali, NaOH for the detection procedure. 200 ml of Sample A was extracted with 20 ml of 2 N NaOH. The alkaline extract of SAMPLE A showed a purple colour extract. The purple colour is typical of 1,4-Diaminoanthraquinone [marker (S)]. The purple colour was detected by the naked eye.

Further, 200 ml of SAMPLE B was extracted with 20 ml of 2 N NaOH. The alkaline extract with SAMPLE B also showed a purple colour extract. The presence of (ethyl 2-[3-(ethylamino)-6-ethylimino-2,7-dimethylxanthen-9-yl]benzoate hydrochloride) [substance (A)] did not affect the detection of 1,4-Diaminoanthraquinone [marker (S)] within the fuel. The purple colour was detected by the naked eye.

Effect of clay on SAMPLE A and SAMPLE B

### Sample A

200 ml of SAMPLE A was shaken with 5g of clay [laundering agent], followed by the addition of 20 ml of 2 N NaOH. The resultant alkaline extract of SAMPLE A showed only a trace detection of purple colour using the naked eye. 1,4-Diaminoanthraquinone [marker (S)] has been removed by the clay [laundering agent].

### Sample B

200 ml of SAMPLE B was shaken with 5g of clay, followed by the addition of 20 ml of 2 N NaOH. The resultant alkaline extract was detected to be purple colour. 1,4-Diaminoanthraquinone [marker (S)] was not completely removed by the clay [laundering agent] in the presence of (ethyl 2-[3-(ethylamino)-6-ethylimino-2,7-dimethylxanthen-9-yl]benzoate hydrochloride) [substance (A)]. The purple colour was detected by the naked eye.

It was therefore concluded that the 1,4-Diaminoanthraquinone [substance (S)] was not completely removed from by the clay in the presence of (ethyl 2-[3-(ethylamino)-6-ethylimino-2,7-dimethylxanthen-9-yl]benzoate hydrochloride) [marker (A)]. i.e. the absorbent properties of the clay has been partially saturated (blocked) by the (ethyl 2-[3-(ethylamino)-6-ethylimino-2,7-dimethylxanthen-9-yl]benzoate hydrochloride) and the 1,4-Diaminoanthraquinone was no longer completely removed.

### Example 2

This example exemplifies the use of Surface-Enhanced Raman Scattering (SERS) spectroscopy for the detection of 1,4-Diaminoanthraquinone [marker (S)] in a particular fuel having 1 parts per million (ppm) of 1,4-Diaminoanthraquinone [marker (S)].

A sample of the fuel marked with 1 ppm of 1,4-Diaminoanthraquinone [marker (S)] was diluted using an unmarked fuel to 10%, i,e, 100 parts per billion (ppb) 1,4-Diaminoanthraquinone [marker (S)]. Subsequently, 0.5ml of colloid (as described in PCT Publication No. WO 2009/081138, which is incorporated herein by reference) was added to 0.5 ml of the diluted sample. The resulting mixture was shaken for 10 to 20 seconds. Following that, 0.1 ml of 0.1M NaCI salt solution was added to the shaken mixture. The sample was then measured on SERS spectrophotometer with a laser set at 532 nm.

### Example 3

This example exemplifies the use of High Performance Liquid Chromatography (HPLC) for the detection of 1,4-Diaminoanthraquinone [marker (S)] in a particular fuel having 1 part per million (ppm) of 1,4-Diaminoanthraquinone [marker (S)].

A 5 ml sample of the fuel having 1 ppm of 1,4-Diaminoanthraquinone [marker (S)] was taken. The marker (S), 1,4-Diaminoanthraquinone, was extracted in 20% ethanoic 2 M HCl acid. The resulting extracted marker was then measured with a fluorescent detector using HPLC (Varian).

### REFERENCES

1. WO 2010/039152

2. U.S. Pat. No. 4,735,631

3. U.S. Pat. No. 4,209,302

4. U.S. Pat. No. 5,525,516

5. U.S. Pat. No. 5,490,872

6. U.S. Pat. No. 5,980,593

7. U.S. Pat. No. 5,156,653

8. WO 2008/019161

9. WO 2009/081138

1.-74. (canceled)

75. A kit for marking, monitoring and/or detecting alteration of a fuel and/or solvent comprising:
(i) at least one marker (S); and
(ii) at least one substance,
wherein the substance is capable of interacting with at least one laundering agent to protect and/or prevent marker (S) from being absorbed, adsorbed, or partially or completely removed by the laundering agent.

76. The kit according to claim 75, wherein the substance is capable of being absorbed, adsorbed and/or partially or completely removed by interaction with at least one laundering agent.

77. The kit according to claim 75, further comprising at least one marker (C), marker (C) being a quantitative marker.

78. The kit according to claim 77, wherein marker (S) is more resistant to at least one laundering agent than marker (C).

79. The kit according to claim 77, wherein the kit comprises
(i) at least one marker (C), at least one marker (S) and at least one substance for addition to a first fuel and/or solvent; and
(ii) at least one marker (C) for addition to a second fuel; both markers (C) being the same.

80. A fuel and/or solvent comprising:
(i) at least one marker (S); and
(ii) at least one substance, wherein the substance is capable of interacting with at least one laundering agent to protect and/or prevent marker (S) from being absorbed, adsorbed, or partially or completely removed by the laundering agent; and
optionally, (iii) at least one marker (C), wherein the marker (C) is a quantitative marker.

81. The fuel and/or solvent according to claim 80, wherein the substance is capable of being absorbed, adsorbed and/or partially or completely removed by interaction with at least one laundering agent.

82. A fuel composition comprising the fuel and/or solvent according to claim 80.

83. The fuel composition according to claim 82, wherein the fuel composition comprises:
a mixture of at least one fuel and/or solvent comprising marker (C), and
a fuel and/or solvent comprising:
(i) at least one marker (S); and
(ii) at least one substance, wherein the substance is capable of interacting with at least one laundering agent to protect and/or prevent marker (S) from being absorbed, adsorbed, or partially or completely removed by the laundering agent; and
optionally, (iii) at least one marker (C), wherein the marker (C) is a quantitative marker.

84. A method of marking fuel and/or solvent, the method comprising:
marking a fuel and/or solvent with:
(i) at least one marker (S); and
(ii) at least one substance,
wherein the substance is capable of interacting with at least one laundering agent to protect and/or prevent marker (S) from being absorbed, adsorbed, or partially or completely removed by the laundering agent.

85. The method according to claim 84, wherein the substance is capable of being absorbed, adsorbed and/or partially or completely removed by interaction with at least one laundering agent.

86. The method according to claim 84, wherein the method further comprises marking the fuel and/or solvent with at least one marker (C), marker (C) being a quantitative marker.

87. The method according to claim 84, wherein the method is a method of marking a first fuel and/or solvent and a second fuel to allow detection of a mixture of the first fuel and second fuel, the method comprising:
(I) marking at least one first fuel and/or solvent with:
(i) at least one marker (S); and
(ii) at least one substance, wherein the substance is capable of interacting with at least one laundering agent; and
(II) marking at least one second fuel with at least one marker (C), wherein marker (C) is a quantitative marker.

88. The method according to claim 87, wherein the method is a method of marking a first fuel and a second fuel to allow detection of a mixture of the first fuel and/or solvent and second fuel, the method comprising:
(I) marking at least one first fuel and/or solvent with:
(i) at least one marker (C), wherein marker (C) is a quantitative marker;
(ii) at least one marker (S); and
(iii) at least one substance, wherein the substance is capable of interacting with at least one laundering agent; and
(II) marking at least one second fuel with at least one marker (C);
both markers (C) being the same.

89. The method according to claim 84, wherein treatment to remove at least some amount of marker (S), results in the partial or complete removal of marker (C) from the fuel.

90. A method of monitoring, testing and/or detecting alteration of a first fuel with a second fuel and/or solvent, the second fuel and/or solvent comprising: (i) at least one marker (S); and (ii) at least one substance, wherein the substance is capable of interacting with at least one laundering agent to protect and/or prevent marker (S) from being absorbed, adsorbed, or partially or completely removed by the laundering agent;
the method comprising:
testing the fuel for the presence of marker (S), wherein the presence of marker (S) in the fuel is indicative that the fuel is altered; or
(II) testing the fuel for the presence of the substance, wherein the substance is capable of being detected, and the presence of the substance is indicative that the fuel is altered; or
(III) testing the fuel for the presence of at least one laundering agent.

91. The method according to claim 90, wherein the substance is capable of being absorbed, adsorbed and/or partially or completely removed by interaction with at least one laundering agent.

92. The method according to claim 90, wherein the method is of monitoring, testing and/or detecting alteration of a first fuel with a second fuel and/or solvent, the first fuel comprising at least one quantitative marker (C), and the second fuel and/or solvent comprising: (i) at least one quantitative marker (C), (ii) at least one marker (S); and (iii) at least one substance, wherein the substance is capable of interacting with at least one laundering agent; both markers (C) being the same and at a predetermined concentration range;
the method further comprising:
testing the fuel for the presence of the quantitative marker (C); wherein the presence of the marker (C) in the fuel having a concentration outside the predetermined concentration range or the absence of a marker (C) in the fuel is indicative that the fuel is altered; or
testing the fuel for the presence of marker (C) within the predetermined concentration range and for the presence of marker (S), wherein the presence of marker (S) in the fuel is indicative that the fuel is altered; or
testing the fuel for the presence of marker (C) within the predetermined concentration range, the absence of marker (S), and the presence of the substance is indicative that the fuel is altered.

93. The method according to claim 90, wherein a treatment to remove marker (S) results in the partial or complete removal of marker (C) from the fuel and/or solvent.

94. A method of monitoring, testing and/or detecting alteration of a fuel, the fuel comprising: (i) at least one marker (S); and (ii) at least one substance, wherein the substance is capable of interacting with at least one laundering agent to protect and/or prevent marker (S) from being absorbed, adsorbed, or partially or completely removed by the laundering agent;
the method comprising:
(I) testing the fuel for the presence of marker (S), wherein the absence of marker (S) in the fuel is indicative that the fuel is altered; or
(II) testing the fuel for the presence of the substance, wherein substance is capable of being detected, the absence of the substance is indicative that the fuel is altered.

95. The method according to claim 94, wherein the fuel further comprises at least one quantitative marker (C) having a predetermined concentration range and is different from marker (S); the method further comprising: testing the fuel for the presence of marker (C) in the fuel, wherein the presence of marker (C) in the fuel having a concentration outside the predetermined concentration range is indicative that the fuel is altered.

96. An apparatus for monitoring, testing and/or detecting alteration of at least one fuel, the fuel and/or solvent comprising: (i) at least one marker (S); and (ii) at least one substance, wherein the substance is capable of interacting with at least one laundering agent to protect and/or prevent marker (S) from being absorbed, adsorbed, or partially or completely removed by the laundering agent; the apparatus comprising at least one means for detecting the presence and/or quantity of at least maker (S) and/or the substance.

97. The apparatus according to claim 96, wherein the fuel and/or solvent further comprises at least one quantitative marker (C).
